(19) United States
(12) Patent Application Publication
(10) Pub. No.: US 2015/0314005 A1
(43) Pub. Date: Nov. 5, 2015

(54) Compositions and methods for amelioration of diseases and related symptoms in humans

(71) Applicant: Jeffrey Alan McKinney, Lafayette, CA (US)

(72) Inventor: Jeffrey Alan McKinney, Lafayette, CA (US)

(21) Appl. No.: 14/545,093

(22) Filed: Mar. 24, 2015

Related U.S. Application Data
(60) Provisional application No. 61/996,256, filed on May 2, 2014.

(51) Int. Cl. A61K 47/46

(52) U.S. Cl. CPC A61K 47/46

(57) ABSTRACT

The present invention is generally directed to compositions and methods for amelioration of diseases and related symptoms in humans. In one aspect, the invention is directed to a method of ameliorating the symptoms of arthritis, brain aging, cataracts, diabetes, dermatitis, chronic renal failure, glaucoma, low energy levels or obesity in a human, wherein the method comprises administering a composition to the animal, wherein the composition comprises a Fruit-Based Additive, a Vegetable-Based Additive or a Fruit- and Vegetable-Based Additive.

### FIELD OF THE INVENTION

The present invention is generally directed to compositions and methods for amelioration of diseases and related symptoms in humans.

### BACKGROUND OF THE INVENTION

There have been reports regarding the treatment of diseases and related symptoms in humans. For example, U.S. Pat. No. 8,609,645, issued Dec. 17, 2013, is allegedly directed to “novel formulations for the treatment of otic infections in an animal comprising a triazole anti-fungal compound, a quinolone antibiotic and a corticosteroid such as mometasone furoate monohydrate.” Abstract.

U.S. Pat. No. 8,710,099, issued Apr. 29, 2014, is allegedly directed to “a method of treating cancer using the sodium salt of a benzoic acid derivative, alone or in combination with standard treatments such as chemotherapy and radiotherapy.” Abstract.

U.S. Pat. No. 8,663,695, issued Mar. 4, 2014, is allegedly directed to “methods and formulations for treating or preventing rhinosinusitis, including fungus-induced rhinosinusitis in mammals. In one embodiment, the formulation of the present invention comprises a steroidal anti-inflammatory agent having a specific particle size distribution profile. The formulation may also comprise an antifungal agent, antibiotic or antiviral agent.” Abstract.

Despite various reports, there is still a need for new compositions and methods for amelioration of diseases and related symptoms in humans.

### SUMMARY OF THE INVENTION

In one aspect, the invention is directed to a method of ameliorating the symptoms of arthritis, brain aging, cataracts, diabetes, dermatitis, glaucoma, low energy levels or obesity in a human, wherein the method comprises administering a composition to the human, wherein the composition comprises a Fruit-Based Additive, a Vegetable-Based Additive or a Fruit- and Vegetable-Based Additive.

### DETAILED DESCRIPTION OF THE INVENTION

The present invention is generally directed to compositions and methods for amelioration of diseases and related symptoms in humans.

“Cesium Salt” refers to a compound that includes a positively charged cesium ion and a negatively charged counterion. Nonlimiting examples of such compounds include: cesium arginate, cesium ascorbate, cesium bicarbonate, cesium caprylate, cesium chloride, cesium cysteinate, cesium citrate, cesium fumarate, cesium humate, cesium fulvate, cesium methionate, cesium glutamate, cesium gluconate, cesium glycinate, cesium aspartate, cesium lysinate, cesium succinate, cesium carbonate, cesium lactate, cesium malate, cesium tartrate, cesium sulfate, cesium phosphate, cesium nitrate, cesium fluoride, cesium bromide, cesium iodide, cesium orotate, and mixtures thereof.

“Dehydrated Food” refers to food materials suitable for feeding that have been dehydrated and which can be reconstituting upon mixing with one or more liquids. The food can include a single ingredient (e.g., chicken or fish) or multiple ingredients. The ingredients can be raw or cooked. Nonlimiting examples of ingredients include: chicken, fish, beef, lamb, venison, eggs, potatoes, sweet potatoes, flaxseed, pumpkin, spinach, cranberries, and mixtures thereof.

“Freeze Drying” refers to preserving something by rapidly freezing it and then subjecting it to a high vacuum that removes ice by sublimation. A preferred freeze drying process includes at least the following steps: macerating fruits and/or vegetables; rinsing the macerated fruits and/or vegetables with chilled water (e.g., 45° C.); macerating the fruits and/or vegetables again; after stabilizing and blending the macerated fruits and/or vegetables, subjecting the mixture to a chill tunnel (e.g., 45° C. to 50° C.); and freeze drying the mixture (e.g., vacuum at approximately −40° C.).

“Fruit-Based Additive” refers to a fruit extract or concentrate. The extract or concentrate includes material from a single or from multiple fruits. One method of making a Fruit-Based Additive concentrate is freeze drying. Nonlimiting examples of fruits that are used to make the additive include dried forms of the following: tomato, banana, cranberry, apple, orange, pomegranate, grape, strawberry, papaya, mango, grape, shitake mushroom and mixtures thereof. In one case, the additive includes the following fruits: orange, papaya/mango, cranberry, grape, strawberry, and shitake mushroom. In another case, the additive includes the following fruits: orange, papaya/mango, cranberry, grape, strawberry, pomegranate, and shitake mushroom.

“Fruit- and Vegetable-Based Additive” refers to a fruit and vegetable extract or concentrate. The extract or concentrate includes material from a single fruit and vegetable or from multiple fruits and vegetables. One method of making a Fruit- and Vegetable-Based Additive concentrate is freeze drying. Nonlimiting examples of fruits and vegetables that are used to make the additive include dried forms of the following: tomato, cranberry, apple, orange, pomegranate, grape, strawberry, papaya, mango, banana, grape, broccoli, carrot, shitake mushrooms, spinach, beet, and mixtures thereof In one case, the additive includes the following fruits and vegetables: tomato, broccoli, carrot, shitake mushrooms, cranberry, apple, and orange. In another case, the additive includes the following fruits and vegetables: cranberry, pomegranate, orange, grape, strawberry, and shitake mushrooms. In still another case, the additive includes the following fruits and vegetables: spinach, broccoli, carrot, tomato, beet, and shitake mushrooms. In another case, the additive includes the following fruits and vegetables: spinach, broccoli, carrot, beet, tomato, shitake mushroom, apple, cranberry, pomegranate, orange, grape and strawberry. In another case, the additive includes the following fruits and vegetables: spinach, broccoli, carrot, beet, tomato, shitake mushroom, apple, cranberry, pomegranate, orange, grape, and strawberry. In another case, the additive includes the following fruits and vegetables: tomato, broccoli, carrot, shitake mushroom, apple, orange, and banana.

“Rubidium Salt” refers to a compound that includes a positively charged rubidium ion and a negatively charged counterion. Nonlimiting examples of such compounds include: rubidium arginate, rubidium ascorbate, rubidium bicarbonate, rubidium caprylate, rubidium fulvate, rubidium methionate, rubidium glutamate, rubidium gluconate, rubidium glycinate, rubidium aspartate, rubidium lysinate, rubidium succinate, rubidium carbonate, rubidium lactate, rubidium malate, rubidium tartrate, rubidium sulfate, rubidium phosphate, rubidium nitrate, rubidium fluoride, rubidium bromide, rubidium iodide, rubidium orotate, and mixtures thereof.

“Symptoms of Arthritis” refers to pain and inflammation in the joints of a human, as well as soft tissue thickening, narrowing of joint spaces, joint effusion (fluid build-up), cartilage changes, bone changes, mineralization of soft tissues, intra-articular calcified bodies (osteophytes) and other physical changes that are known to be associated with arthritis. Radiographs, commonly called X-rays, are a very effective tool for identifying and assessing arthritis.

“Arthritis Nutritional” refers to one or more of the following: chondroitin sulfate; glucosamine sulfate; calcium; vitamin D3; ginger, turmeric; omega-3; green tea; SAMe; MSM; stinging nettle; bromelian; vitamin E; and, Devil's claw.

“Symptoms of Brain Aging” refers to a disturbance of activity and sleep patterns, an increase in aimless wandering and pacing, compulsive behaviors (e.g., circling, tremors, stiffness and weakness). Brain aging is often diagnosed as the result of a neurological examination.

“Brain Aging Nutritional” refers to one or more of the following: vitamin B1; vitamin C; vitamin D; vitamin E; beta-carotene; ginseng; green tea extract; and, cod liver oil.

“Symptoms of Cataracts” refers to cloudy, white-ish or blue-grey pupils. Cataracts are oftentimes diagnosed by dilation of pupils and evaluation of cataract extent.

“Cataract Nutritional” refers to one or more of the following: vitamin C; Vitamin E; bilberries; beta-carotene; astaxanthin; glutathione; alpha-lipoic acid.

“Symptoms of Conjunctivitis” refers to squinting or spasmodic blinking (blephora), redness of the moist tissues of the eye, discharge from the eye(s), swelling of the moist tissue covering the eyeball, follicle formation. Conjunctivitis is oftentimes diagnosed using one or more of the following tests: culture of eye discharge; and, biopsy of conjunctiva cells.

“Conjunctivitis Nutritional” refers to one or more of the following: vitamin A; vitamin C; zinc; eyebright; chamomile; licorice root; and, turmeric.

“Symptoms of Diabetes” refers to frequent urination and excessive thirst. Diabetes is oftentimes diagnosed using one or more of the following tests: a blood test.

“Diabetes Nutritional” refers to one or more of the following: chromium; vitamin E; selenium; acarbose.

“Symptoms of Dermatitis” refers to itchy red bumps, skin inflammation, blisters, crusts, and hair loss.

“Dermatitis Nutritional” refers to one or more of the following: omega-3 fatty acids (EPA and DHA); omega-6 fatty acids (linoleic acid); vitamin C; vitamin E; vitamin A; zinc; reishi mushrooms; echinacea; astragulus root; and, thymus.

“Symptoms of Glaucoma” refers to high pressure within the eye, blinking of the eye, recession of the eyeball into the head, redness of blood vessels in the whites of the eyes, cloudy appearance at the front of the eye, dilated pupils, vision loss. Glaucoma is oftentimes diagnosed according to the elevated pressure within a human's eyes.

“Glaucoma Nutritional” refers to vitamin A, vitamin C, vitamin E, bilberry extract, zinc, lutein, quercetin, alpha lipoic acid, mixed carotenoids, and grape seed extract.

“Symptoms of Lower Energy Level” refers to eating and/or drinking less than normal, walking with difficulty, lethargy.

“Energy Level Nutritional” refers to one or more of the following: eleuthero; rosemary; spirulina; amalaki; vitamin B; vitamin B12; coQ10; and, PQQ.

“Symptoms of Obesity” refers to weight gain, excess body fat, unwillingness to exercise.

“Obesity Nutritional” refers to one or more of the following: fiber, chromium picolinate, essential fatty acids, kelp, lecithin, spirulina, vitamin C, and yerba mate.

“Symptoms of Chronic Renal Failure” refers to nausea, vomiting, loss of appetite, fatigue and weakness, sleep problems, changes in urine output, decreased mental sharpness, and muscle twitches and cramps.

“Chronic Renal Failure Nutritional” refers to one or more of the following: vitamin A, vitamin D, vitamin E, vitamin K, vitamin B1, vitamin B2, niacin, vitamin B6, folate, vitamin B12, vitamin C, and pantothenic acid.

“Vegetable-Based Additive” refers to a vegetable extract or concentrate. The extract includes material from a single vegetable or from multiple vegetables. One method of making a Vegetable-Based Additive concentrate is freeze drying. Nonlimiting examples of vegetables that are used to make the additive include dried forms of the following: broccoli, carrot, shitake mushrooms, spinach, beet, and mixtures thereof. In one case, the additive includes the following vegetables: spinach, broccoli, carrot, tomato, beet, and shitake mushroom. In another case, the additive includes the following vegetables: spinach, broccoli, carrot, tomato, beet and shitake mushroom.

The compositions and methods of the present invention are directed to the amelioration of symptoms related to arthritis, brain aging, cataracts, conjunctivitis, diabetes, dermatitis, glaucoma, low energy levels, chronic renal failure, and obesity in humans.

In one case, compositions according to the present invention comprise a Cesium Salt and/or a Rubidium Salt.

In another case, compositions according to the present invention comprise a Fruit-Based, Vegetable-Based, or a Fruit- and Vegetable-Based Additive. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

In another case, compositions according to the present invention comprise Dehydrated Food and a Fruit-Based Additive, Dehydrated Food and a Vegetable-Based Additive, or Dehydrated Food and a Fruit- and Vegetable-Based Additive.

In another case, compositions according to the present invention comprise a Fruit-Based Additive and one or more additives selected from the following: chondroitin sulfate; glucosamine sulfate; calcium; vitamin D3; ginger, turmeric; omega-3; green tea; SAMe; MSM; stinging nettle; bromelian; vitamin E; and, Devil's claw. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom.

In another case, compositions according to the present invention comprise a Fruit-Based Additive and one or more additives selected from the following: vitamin B1; vitamin C; vitamin D; vitamin E; beta-carotene; ginseng; green tea extract; and, cod liver oil. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom.

In another case, compositions according to the present invention comprise a Fruit-Based Additive and one or more additives selected from the following: vitamin C; Vitamin E; bilberries; beta-carotene; astaxanthin; glutathione; alpha-lipoic acid. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom.

In another case, compositions according to the present invention comprise a Fruit-Based Additive and one or more additives selected from the following: vitamin A; vitamin C; zinc; eyebright; chamomile; licorice root; and, turmeric. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom.

In another case, compositions according to the present invention comprise a Fruit-Based Additive and one or more additives selected from the following: chromium; vitamin E; selenium; acarbose. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom.

In another case, compositions according to the present invention comprise a Fruit-Based Additive and one or more additives selected from the following: omega-3 fatty acids (EPA and DHA); omega-6 fatty acids (linoleic acid); vitamin C; vitamin E; vitamin A; zinc; reishi mushrooms; echinacea; astragulus root; and, thymus. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom.

In another case, compositions according to the present invention comprise a Fruit-Based Additive and one or more additives selected from the following: glucosamine; chondroitin; and, cosequin. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom.

In another case, compositions according to the present invention comprise a Fruit-Based Additive and one or more additives selected from the following: vitamin A, vitamin C, vitamin E, bilberry extract, zinc, lutein, quercetin, alpha lipoic acid, mixed carotenoids, and grape seed extract. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom.

In another case, compositions according to the present invention comprise a Fruit-Based Additive and one or more additives selected from the following: eleuthero; rosemary; spirulina; amalaki; vitamin B; vitamin B12; coQ10; and, PQQ. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom.

In another case, compositions according to the present invention comprise a Fruit-Based Additive and one or more additives selected from the following: fiber, chromium picolinate, essential fatty acids, kelp, lecithin, spirulina, vitamin C, and yerba mate. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom.

In another case, compositions according to the present invention comprise a Fruit-Based Additive and one or more additives selected from the following: vitamin A, vitamin D, vitamin E, vitamin K, vitamin B1, vitamin B2, niacin, vitamin B6, folate, vitamin B12, vitamin C, and pantothenic acid. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom.

In another case, compositions according to the present invention comprise a Vegetable-Based Additive and one or more additives selected from the following: chondroitin sulfate; glucosamine sulfate; calcium; vitamin D3; ginger, turmeric; omega-3; green tea; SAMe; MSM; stinging nettle; bromelian; vitamin E; and, Devil's claw. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom.

In another case, compositions according to the present invention comprise a Vegetable-Based Additive and one or more additives selected from the following: vitamin B1; vitamin C; vitamin D; vitamin E; beta-carotene; ginseng; green tea extract; and, cod liver oil. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom.

In another case, compositions according to the present invention comprise a Vegetable-Based Additive and one or more additives selected from the following: vitamin C; Vitamin E; bilberries; beta-carotene; astaxanthin; glutathione; alpha-lipoic acid. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom.

In another case, compositions according to the present invention comprise a Vegetable-Based Additive and one or more additives selected from the following: vitamin A; vitamin C; zinc; eyebright; chamomile; licorice root; and, turmeric. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom.

In another case, compositions according to the present invention comprise a Vegetable-Based Additive and one or more additives selected from the following: chromium; vitamin E; selenium; acarbose. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom.

In another case, compositions according to the present invention comprise a Vegetable-Based Additive and one or more additives selected from the following: omega-3 fatty acids (EPA and DHA); omega-6 fatty acids (linoleic acid); vitamin C; vitamin E; vitamin A; zinc; reishi mushrooms; echinacea; astragulus root; and, thymus. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom.

In another case, compositions according to the present invention comprise a Vegetable-Based Additive and one or more additives selected from the following: glucosamine; chondroitin; and, cosequin. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom.

In another case, compositions according to the present invention comprise a Vegetable-Based Additive and one or more additives selected from the following: vitamin A, vitamin C, vitamin E, bilberry extract, zinc, lutein, quercetin, alpha lipoic acid, mixed carotenoids, and grape seed extract. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom.

In another case, compositions according to the present invention comprise a Vegetable-Based Additive and one or more additives selected from the following: eleuthero; rosemary; spirulina; amalaki; vitamin B; vitamin B12; coQ10; and, PQQ. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom.

In another case, compositions according to the present invention comprise a Vegetable-Based Additive and one or more additives selected from the following: fiber, chromium picolinate, essential fatty acids, kelp, lecithin, spirulina, vitamin C, and yerba mate. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom.

In another case, compositions according to the present invention comprise a Vegetable-Based Additive and one or more additives selected from the following: vitamin A, vitamin D, vitamin E, vitamin K, vitamin B1, vitamin B2, niacin, vitamin B6, folate, vitamin B12, vitamin C, and pantothenic acid. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom.

In another case, compositions according to the present invention comprise a Fruit- and Vegetable-Based Additive and one or more additives selected from the following: chondroitin sulfate; glucosamine sulfate; calcium; vitamin D3; ginger, turmeric; omega-3; green tea; SAMe; MSM; stinging nettle; bromelian; vitamin E; and, Devil's claw. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. hi another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

In another case, compositions according to the present invention comprise a Fruit- and Vegetable-Based Additive and one or more additives selected from the following: vitamin B1; vitamin C; vitamin D; vitamin E; beta-carotene; ginseng; green tea extract; and, cod liver oil. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

In another case, compositions according to the present invention comprise a Fruit- and Vegetable-Based Additive and one or more additives selected from the following: vitamin C; Vitamin E; bilberries; beta-carotene; astaxanthin; glutathione; alpha-lipoic acid. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

In another case, compositions according to the present invention comprise a Fruit- and Vegetable-Based Additive and one or more additives selected from the following: vitamin A; vitamin C; zinc; eyebright; chamomile; licorice root; and, turmeric. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

In another case, compositions according to the present invention comprise a Fruit- and Vegetable-Based Additive and one or more additives selected from the following: chromium; vitamin E; selenium; acarbose. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

In another case, compositions according to the present invention comprise a Fruit- and Vegetable-Based Additive and one or more additives selected from the following: omega-3 fatty acids (EPA and DHA); omega-6 fatty acids (linoleic acid); vitamin C; vitamin E; vitamin A; zinc; reishi mushrooms; echinacea; astragulus root; and, thymus. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

In another case, compositions according to the present invention comprise a Fruit- and Vegetable-Based Additive and one or more additives selected from the following: glucosamine; chondroitin; and, cosequin. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

In another case, compositions according to the present invention comprise a Fruit- and Vegetable-Based Additive and one or more additives selected from the following: vitamin A, vitamin C, vitamin E, bilberry extract, zinc, lutein, quercetin, alpha lipoic acid, mixed carotenoids, and grape seed extract. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

In another case, compositions according to the present invention comprise a Fruit- and Vegetable-Based Additive and one or more additives selected from the following: eleuthero; rosemary; spirulina; amalaki; vitamin B; vitamin B12; coQ10; and, PQQ. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

In another case, compositions according to the present invention comprise a Fruit- and Vegetable-Based Additive and one or more additives selected from the following: fiber, chromium picolinate, essential fatty acids, kelp, lecithin, spirulina, vitamin C, and yerba mate. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

In another case, compositions according to the present invention comprise a Fruit- and Vegetable-Based Additive and one or more additives selected from the following: vitamin A, vitamin D, vitamin E, vitamin K, vitamin B1, vitamin B2, niacin, vitamin B6, folate, vitamin B12, vitamin C, and pantothenic acid. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

In one aspect, the present invention is directed to the amelioration of symptoms related to arthritis in humans. The method comprises administering a composition to the human, wherein the composition is selected from a group of compositions, where the compositions comprise the listed ingredients/additives:

A Cesium Salt and/or a Rubidium Salt;

A Fruit-Based, Vegetable-Based, or a Fruit- and Vegetable-Based Additive. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana;

Dehydrated Food and a Fruit-Based Additive, Dehydrated Food and a Vegetable-Based Additive, or Dehydrated Food and a Fruit- and Vegetable-Based Additive;

A Fruit-Based Additive and one or more additives selected from the following: chondroitin sulfate; glucosamine sulfate; calcium; vitamin D3; ginger, turmeric; omega-3; green tea; SAMe; MSM; stinging nettle; bromelian; vitamin E; and, Devil's claw. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom;

A Vegetable-Based Additive and one or more additives selected from the following: chondroitin sulfate; glucosamine sulfate; calcium; vitamin D3; ginger, turmeric; omega-3; green tea; SAMe; MSM; stinging nettle; bromelian; vitamin E; and, Devil's claw. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom;

A Fruit- and Vegetable-Based Additive and one or more additives selected from the following: chondroitin sulfate; glucosamine sulfate; calcium; vitamin D3; ginger, turmeric; omega-3; green tea; SAMe; MSM; stinging nettle; bromelian; vitamin E; and, Devil's claw. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

In another aspect, the present invention is directed to the amelioration of symptoms related to brain aging in humans. The method comprises administering a composition to the human, wherein the composition is selected from a group of compositions, where the compositions comprise the listed ingredients/additives:

A Cesium Salt and/or a Rubidium Salt;

A Fruit-Based, Vegetable-Based, or a Fruit- and Vegetable-Based Additive. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana;

Dehydrated Food and a Fruit-Based Additive, Dehydrated Food and a Vegetable-Based Additive, or Dehydrated Food and a Fruit- and Vegetable-Based Additive;

A Fruit-Based Additive and one or more additives selected from the following: vitamin B1; vitamin C; vitamin D; vitamin E; beta-carotene; ginseng; green tea extract; and, cod liver oil. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom;

A Vegetable-Based Additive and one or more additives selected from the following: vitamin B1; vitamin C; vitamin D; vitamin E; beta-carotene; ginseng; green tea extract; and, cod liver oil. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom;

A Fruit- and Vegetable-Based Additive and one or more additives selected from the following: vitamin B1; vitamin C; vitamin D; vitamin E; beta-carotene; ginseng; green tea extract; and, cod liver oil. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

In another aspect, the present invention is directed to the amelioration of symptoms related to cataracts in humans. The method comprises administering a composition to the human, wherein the composition is selected from a group of compositions, where the compositions comprise the listed ingredients/additives:

A Cesium Salt and/or a Rubidium Salt;

A Fruit-Based, Vegetable-Based, or a Fruit- and Vegetable-Based Additive. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana;

Dehydrated Food and a Fruit-Based Additive, Dehydrated Food and a Vegetable-Based Additive, or Dehydrated Food and a Fruit- and Vegetable-Based Additive;

A Fruit-Based Additive and one or more additives selected from the following: vitamin C; Vitamin E; bilberries; beta-carotene; astaxanthin; glutathione; alpha-lipoic acid. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom;

A Vegetable-Based Additive and one or more additives selected from the following: vitamin C; Vitamin E; bilberries; beta-carotene; astaxanthin; glutathione; alpha-lipoic acid. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom;

A Fruit- and Vegetable-Based Additive and one or more additives selected from the following: vitamin C; Vitamin E; bilberries; beta-carotene; astaxanthin; glutathione; alpha-lipoic acid. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

In another aspect, the present invention is directed to the amelioration of symptoms related to conjunctivitis in humans. The method comprises administering a composition to the human, wherein the composition is selected from a group of compositions, where the compositions comprise the listed ingredients/additives:

A Cesium Salt and/or a Rubidium Salt;

A Fruit-Based, Vegetable-Based, or a Fruit- and Vegetable-Based Additive. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana;

Dehydrated Food and a Fruit-Based Additive, Dehydrated Food and a Vegetable-Based Additive, or Dehydrated Food and a Fruit- and Vegetable-Based Additive;

A Fruit-Based Additive and one or more additives selected from the following: vitamin A; vitamin C; zinc; eyebright; chamomile; licorice root; and, turmeric. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom;

A Vegetable-Based Additive and one or more additives selected from the following: vitamin A; vitamin C; zinc; eyebright; chamomile; licorice root; and, turmeric. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom;

A Fruit- and Vegetable-Based Additive and one or more additives selected from the following: vitamin A; vitamin C; zinc; eyebright; chamomile; licorice root; and, turmeric. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

In another aspect, the present invention is directed to the amelioration of symptoms related to diabetes in humans. The method comprises administering a composition to the human, wherein the composition is selected from a group of compositions, where the compositions comprise the listed ingredients/additives:

A Cesium Salt and/or a Rubidium Salt;

A Fruit-Based, Vegetable-Based, or a Fruit- and Vegetable-Based Additive. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana;

Dehydrated Food and a Fruit-Based Additive, Dehydrated Food and a Vegetable-Based Additive, or Dehydrated Food and a Fruit- and Vegetable-Based Additive;

A Fruit-Based Additive and one or more additives selected from the following: chromium; vitamin E; selenium; acarbose. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom;

A Vegetable-Based Additive and one or more additives selected from the following: chromium; vitamin E; selenium; acarbose. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom;

A Fruit- and Vegetable-Based Additive and one or more additives selected from the following: chromium; vitamin E; selenium; acarbose. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable- Vegetable-Based Additive includes the following fruits and vegetables: tomato, broccoli, carrot, shitake mushroom, apple, orange, and banana.

In another aspect, the present invention is directed to the amelioration of symptoms related to dermatitis in humans. The method comprises administering a composition to the human, wherein the composition is selected from a group of compositions, where the compositions comprise the listed ingredients/additives:

A Cesium Salt and/or a Rubidium Salt;

A Fruit-Based, Vegetable-Based, or a Fruit- and Vegetable-Based Additive. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana;

Dehydrated Food and a Fruit-Based Additive, Dehydrated Food and a Vegetable-Based Additive, or Dehydrated Food and a Fruit- and Vegetable-Based Additive;

A Fruit-Based Additive and one or more additives selected from the following: omega-3 fatty acids (EPA and DHA); omega-6 fatty acids (linoleic acid); vitamin C; vitamin E; vitamin A; zinc; reishi mushrooms; echinacea; astragulus root; and, thymus. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom;

A Vegetable-Based Additive and one or more additives selected from the following: omega-3 fatty acids (EPA and DHA); omega-6 fatty acids (linoleic acid); vitamin C; vitamin E; vitamin A; zinc; reishi mushrooms; echinacea; astragulus root; and, thymus. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom;

A Fruit- and Vegetable-Based Additive and one or more additives selected from the following: omega-3 fatty acids (EPA and DHA); omega-6 fatty acids (linoleic acid); vitamin C; vitamin E; vitamin A; zinc; reishi mushrooms; echinacea; astragulus root; and, thymus. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

In another aspect, the present invention is directed to the amelioration of symptoms related to glaucoma in humans. The method comprises administering a composition to the human, wherein the composition is selected from a group of compositions, where the compositions comprise the listed ingredients/additives:

A Cesium Salt and/or a Rubidium Salt;

A Fruit-Based, Vegetable-Based, or a Fruit- and Vegetable-Based Additive. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana;

Dehydrated Food and a Fruit-Based Additive, Dehydrated Food and a Vegetable-Based Additive, or Dehydrated Food and a Fruit- and Vegetable-Based Additive;

A Fruit-Based Additive and one or more additives selected from the following: vitamin A, vitamin C, vitamin E, bilberry extract, zinc, lutein, quercetin, alpha lipoic acid, mixed carotenoids, and grape seed extract. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom;

A Vegetable-Based Additive and one or more additives selected from the following: vitamin A, vitamin C, vitamin E, bilberry extract, zinc, lutein, quercetin, alpha lipoic acid, mixed carotenoids, and grape seed extract. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom;

A Fruit- and Vegetable-Based Additive and one or more additives selected from the following: vitamin A, vitamin C, vitamin E, bilberry extract, zinc, lutein, quercetin, alpha lipoic acid, mixed carotenoids, and grape seed extract. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

In another aspect, the present invention is directed to the amelioration of symptoms related to low energy levels in humans. The method comprises administering a composition to the human, wherein the composition is selected from a group of compositions, where the compositions comprise the listed ingredients/additives:

A Cesium Salt and/or a Rubidium Salt;

A Fruit-Based, Vegetable-Based, or a Fruit- and Vegetable-Based Additive. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana;

Dehydrated Food and a Fruit-Based Additive, Dehydrated Food and a Vegetable-Based Additive, or Dehydrated Food and a Fruit- and Vegetable-Based Additive;

A Fruit-Based Additive and one or more additives selected from the following: eleuthero; rosemary; spirulina; amalaki; vitamin B; vitamin B12; coQ10; and, PQQ. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom;

A Vegetable-Based Additive and one or more additives selected from the following: eleuthero; rosemary; spirulina; amalaki; vitamin B; vitamin B12; coQ10; and, PQQ. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom;

A Fruit- and Vegetable-Based Additive and one or more additives selected from the following: eleuthero; rosemary; spirulina; amalaki; vitamin B; vitamin B12; coQ10; and, PQQ. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

In another aspect, the present invention is directed to the amelioration of symptoms related to obesity in humans. The method comprises administering a composition to the human, wherein the composition is selected from a group of compositions, where the compositions comprise the listed ingredients/additives:

A Cesium Salt and/or a Rubidium Salt;

A Fruit-Based, Vegetable-Based, or a Fruit- and Vegetable-Based Additive. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana;

Dehydrated Food and a Fruit-Based Additive, Dehydrated Food and a Vegetable-Based Additive, or Dehydrated Food and a Fruit- and Vegetable-Based Additive;

A Fruit-Based Additive and one or more additives selected from the following: fiber, chromium picolinate, essential fatty acids, kelp, lecithin, spirulina, vitamin C, and yerba mate. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom;

A Vegetable-Based Additive and one or more additives selected from the following: fiber, chromium picolinate, essential fatty acids, kelp, lecithin, spirulina, vitamin C, and yerba mate. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom;

A Fruit- and Vegetable-Based Additive and one or more additives selected from the following: fiber, chromium picolinate, essential fatty acids, kelp, lecithin, spirulina, vitamin C, and yerba mate. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

In another aspect, the present invention is directed to the amelioration of symptoms related to chronic renal failure in humans. The method comprises administering a composition to the human, wherein the composition is selected from a group of compositions, where the compositions comprise the listed ingredients/additives:

A Cesium Salt and/or a Rubidium Salt;

A Fruit-Based, Vegetable-Based, or a Fruit- and Vegetable-Based Additive. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana;

Dehydrated Food and a Fruit-Based Additive, Dehydrated Food and a Vegetable-Based Additive, or Dehydrated Food and a Fruit- and Vegetable-Based Additive;

A Fruit-Based Additive and one or more additives selected from the following: vitamin A, vitamin D, vitamin E, vitamin K, vitamin B1, vitamin B2, niacin, vitamin B6, folate, vitamin B12, vitamin C, and pantothenic acid. In one composition, the Fruit-Based Additive includes the following fruits: orange, papaya/mango, cranberry, grape, strawberry, and shitake mushroom. In another composition, the Fruit-Based Additive includes the following fruits: orange, papaya/mango, cranberry, grape, strawberry, pomegranate, and shitake mushroom;

A Vegetable-Based Additive and one or more additives selected from the following: vitamin A, vitamin D, vitamin E, vitamin K, vitamin B1, vitamin B2, niacin, vitamin B6, folate, vitamin B12, vitamin C, and pantothenic acid. In one composition, the Vegetable-Based Additive includes the following vegetables: spinach, broccoli, carrot, tomato, beet, and shitake mushroom. In another composition, the Vegetable-Based Additive includes the following vegetables: spinach, broccoli, carrot, tomato, beet and shitake mushroom;

A Fruit- and Vegetable-Based Additive and one or more additives selected from the following: vitamin A, vitamin D, vitamin E, vitamin K, vitamin B1, vitamin B2, niacin, vitamin B6, folate, vitamin B12, vitamin C, and pantothenic acid. In one composition the Fruit- and Vegetable-Based Additive includes the following fruits and vegetables: tomato, broccoli, carrot, shitake mushrooms, cranberry, apple, and orange. In another composition, the Fruit- and Vegetable-Based Additive includes the following fruits and vegetables: cranberry, pomegranate, orange, grape, strawberry, and shitake mushrooms. In still another composition, the Fruit- and Vegetable-Based Additive includes the following fruits and vegetables: spinach, broccoli, carrot, tomato, beet, and shitake mushrooms. In another composition, the Fruit- and Vegetable-Based Additive includes the following fruits and vegetables: spinach, broccoli, carrot, beet, tomato, shitake mushroom, apple, cranberry, pomegranate, orange, grape and strawberry. In another composition, the Fruit- and Vegetable-Based Additive includes the following fruits and vegetables: spinach, broccoli, carrot, beet, tomato, shitake mushroom, apple, cranberry, pomegranate, orange, grape, and strawberry. In another composition, the Fruit- and Vegetable-Based Additive includes the following fruits and vegetables: tomato, broccoli, carrot, shitake mushroom, apple, orange, and banana.

Where one or more cesium and/or rubidium salts are in the composition used to ameliorate symptoms of a human, the amount of salt included typically ranges from one milligram to one gram. The included amount is suitably varied to obtain a desired level of symptom reduction.

Where Fruit-, Vegetable- or Fruit- and Vegetable-Based additives are in the composition used to ameliorate symptoms of a human, the amount of additive included typically ranges from one milligram to ten grams. The included amount is suitably varied to obtain a desired level of symptom reduction.

Where Dehydrated Food is in the composition used to ameliorate symptoms of a human, the amount of dehydrated food included typically ranges from 100 milligrams to one kilogram. The included amount is suitably varied to obtain a desired level of symptom reduction.

Where additives other than the above are in the composition used to ameliorate symptoms of a human, the amount included typically ranges from one milligram to 10 grams. The included amount is suitably varied to obtain a desired level of symptom reduction.

1. A method of ameliorating the symptoms of arthritis, brain aging, cataracts, diabetes, dermatitis, glaucoma, low energy levels, chronic renal failure, or obesity in a human, wherein the method comprises administering a composition to the human, wherein the composition comprises a Fruit-Based Additive, a Vegetable-Based Additive or a Fruit- and Vegetable-Based Additive.
